(19) United States
(12) Patent Application Publication
(10) Pub. No.: US 2007/0250151 A1
(43) Pub. Date: Oct. 25, 2007

(54) Endovascular aortic repair delivery system with anchor

(75) Inventor: Peter J. Pereira, Revere, MA (US)

Correspondence Address: RATNER PRESTIA, P.O. BOX 980, VALLEY FORGE, PA, 19482, US

(73) Assignee: Scimed Life Systems, Inc.

(21) Appl. No.: 11/409,579

(22) Filed: Apr. 24, 2006

(51) Int. Cl. A61F 2/84

(52) U.S. Cl. 623/1.12

(57) ABSTRACT

A delivery system is provided for deploying a prosthesis in a body lumen, the prosthesis having a radially compressed configuration. The delivery system includes a primary elongated member positioned coaxially within the prosthesis. A secondary elongated member surrounds a portion of the primary elongated member and a portion of the secondary elongated member is positioned coaxially within the prosthesis. The delivery system further includes a proximal anchor attached to the secondary elongated member. The anchor is adapted for engagement with the proximal end of the prosthesis, thereby maintaining the prosthesis in its radially compressed configuration. At least one outer sheath is adapted to be retracted to expose the prosthesis while the prosthesis is maintained in its radially compressed configuration. The primary elongated member and the secondary elongated member are axially movable relative to one another to disengage the prosthesis from the anchor and permit expansion of the radially compressed prosthesis.

### BACKGROUND OF THE INVENTION

Endovascular Aortic Repair (EVAR) delivery systems typically delivers a prosthesis by a sheath retraction mechanism in which the prosthesis is held in place by a stabilizer within the delivery system while the sheath is being retracted. A conventional EVAR delivery system thus typically transmits a compressive force to the prosthesis during deployment. Such a compressive force adds to the force required to retract the sheath and expose the prosthesis.

Accordingly, there remains a need for an EVAR delivery system that minimizes compressive forces on the prosthesis and provides smooth delivery and accurate positioning of the prosthesis in the vasculature.

### SUMMARY OF THE INVENTION

A delivery system is provided for deploying a prosthesis in a body lumen, the prosthesis having a proximal end, a distal end, and a radially compressed configuration. As used herein, the term “proximal” refers to the end closer to an access location outside the body, whereas “distal” refers to the end farther from the access location. The delivery system has a proximal end and a distal end, and includes a primary elongated member positioned coaxially within the prosthesis and having a proximal end and a distal end. A secondary elongated member surrounds a portion of the primary elongated member and a portion of the secondary elongated member is positioned coaxially within the prosthesis. The secondary elongated member has a proximal end and a distal end. The delivery system further includes a proximal anchor attached to the secondary elongated member. The anchor is adapted for engagement with the proximal end of the prosthesis, thereby maintaining the prosthesis in its radially compressed configuration. At least one outer sheath is adapted to be retracted to expose the prosthesis while the prosthesis is maintained in its radially compressed configuration. The primary elongated member and the secondary elongated member are axially movable relative to one another to disengage the prosthesis from the anchor and permit expansion of the radially compressed prosthesis.

### BRIEF DESCRIPTION OF THE DRAWINGS

FIG. 1A is a plan view of a delivery system for deploying a prosthesis in a body lumen, shown with an outer sheath retracted to expose the prosthesis while the prosthesis is maintained in its radially compressed configuration;

FIG. 1B is the delivery system as illustrated in FIG. 1A shown with the proximal end of the prosthesis expanded and the distal end of the prosthesis maintained in its radially compressed configuration;

FIG. 1C is the delivery system as illustrated in FIG. 1A shown with the proximal end of the prosthesis expanded and the distal end of the prosthesis expanded;

FIG. 2A is a plan view of another delivery system for deploying a prosthesis in a body lumen, shown with a proximal outer sheath and a distal outer sheath mated together while the prosthesis is maintained in its radially compressed configuration;

FIG. 2B is the delivery system as illustrated in FIG. 2A shown with the distal end of the prosthesis expanded and the proximal end of the prosthesis maintained in its radially compressed configuration; and

FIG. 2C is the delivery system as illustrated in FIG. 2A shown with the proximal end of the prosthesis expanded and the distal end of the prosthesis expanded.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

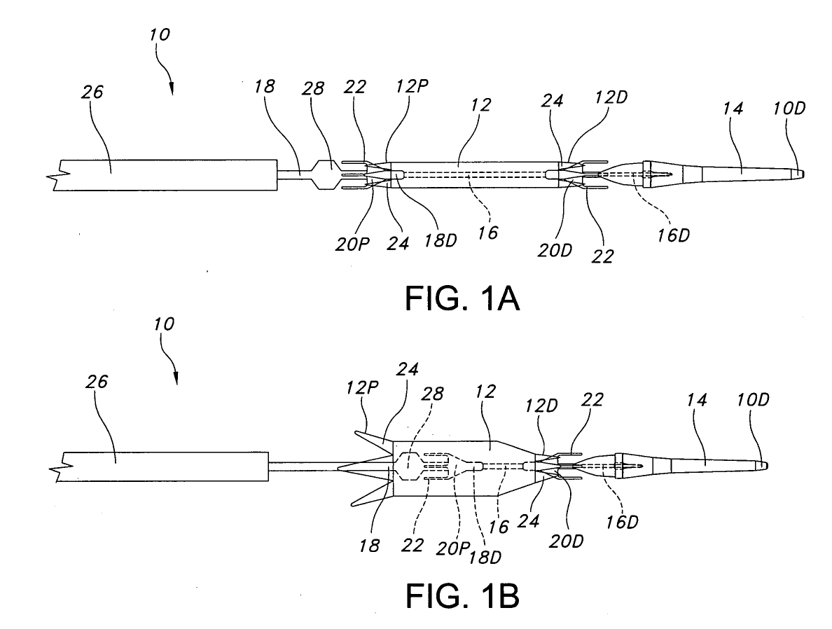

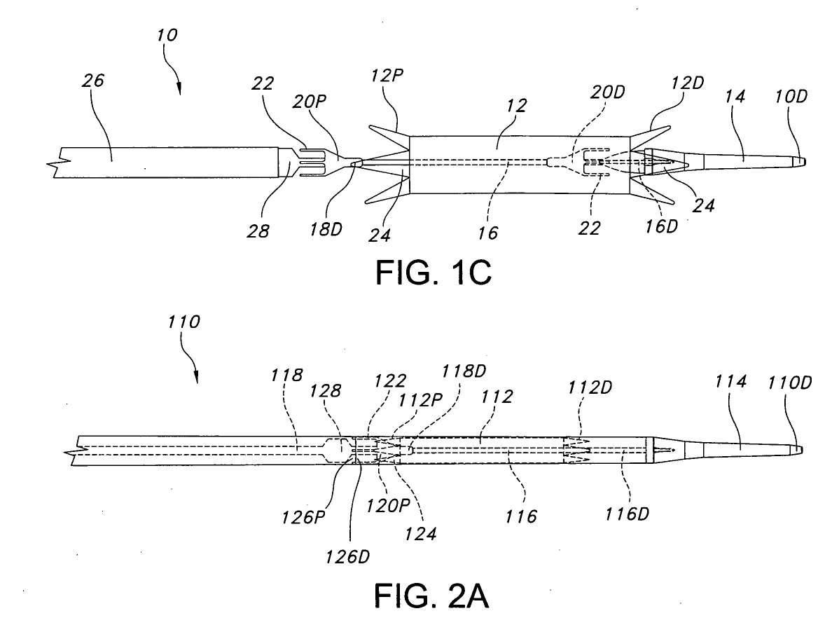

Referring generally to FIGS. 1A-1C, there is shown a delivery system 10 for deploying a prosthesis 12 in a body lumen (not shown), prosthesis 12 having a proximal end 12P, a distal end 12D, and a radially compressed configuration, as illustrated in FIG. 1A. Delivery system 10 has a proximal end (not shown) and a distal end 10D. A tip 14 is attached or over molded at the distal end 10D of delivery system 10, and a pusher handle (not shown) is located at the proximal end and remains outside the body lumen.

Delivery system 10 includes a primary elongated member 16 positioned coaxially within prosthesis 12 and having a proximal end (not shown) and a distal end 16D. A secondary elongated member 18 surrounds a portion of primary elongated member 16 and a portion of secondary elongated member 18 is positioned coaxially within prosthesis 12. Secondary elongated member 18 has a proximal end (not shown) and a distal end 18D.

Delivery system 10 further includes a proximal anchor 20P attached to secondary elongated member 18, and a distal anchor 20D attached to primary elongated member 16. Proximal anchor 20P is adapted for engagement with the proximal end of prosthesis 12P, and distal anchor 20D is adapted for engagement with the distal end of prosthesis 12D, thereby maintaining prosthesis 12 in its radially compressed configuration. More specifically in the embodiment shown in FIGS. 1A-1C, each of proximal anchor 20P and distal anchor 20D includes prongs 22 adapted for engagement with apertures 24 in prosthesis 12. Prongs 22 of proximal anchor 20P extend toward the proximal end of delivery system 10, and prongs 22 of distal anchor 20D extend toward the distal end 10D of delivery system 10.

Prosthesis 12 may consist of, among other things, a self-expanding stent or a self-expanding stent-graft (as represented in FIGS. 1A-1C). End portions 12P and 12D of stent-graft 12 represent wire end loops of the stent that are not covered by the graft. For clarity purposes, the stent portion covered by the graft of stent-graft 12 is not shown. Apertures 24 represent the openings within the wire end loops of the stent. As illustrated in FIG. 1A, prongs 22 of proximal anchor 20P and distal anchor 20D are hooked through (i.e., engaged with) openings 24 within wire end loops 12P and 12D, respectively, of the stent of prosthesis 12. Such engagement of anchors 20P, 20D with ends 12P, 12D, respectively, of prosthesis 12, maintains prosthesis 12 in its radially compressed configuration. In other words, prongs 22 of anchors 20P, 20D effectively grab the ends 12P, 12D of prosthesis 12 to prevent prosthesis 12 from self-expanding.

An outer sheath 26 is adapted to be retracted to expose prosthesis 12 while prosthesis 12 is maintained in its radially compressed configuration under tension between anchors 20P, 20D, as illustrated in FIG. 1A. Holding the prosthesis under tension minimizes radial forces exerted on outer sheath 26 by the self-expanding stent and thus minimizes the frictional force between prosthesis 12 and outer sheath 26 that adds to the force required to retract outer sheath 26 and expose prosthesis 12.

Primary elongated member 16 and secondary elongated member 18 are axially movable relative to one another to disengage prosthesis 12 from anchors 20P, 20D and permit expansion of the radially compressed prosthesis 12, as illustrated in FIG. 1C. In one embodiment, each of primary elongated member 16 and secondary elongated member 18 comprises a hypotube or single lumen extrusion. Primary elongated member 16 may guide delivery system 10 through the body lumen (not shown) over a guidewire (not shown) to the area to be repaired.

For clarity purposes, primary elongated member 16 and secondary elongated member 18 are not represented (with hidden lines) within outer sheath 26. It is to be understood, however, that secondary elongated member 18 extends proximally within outer sheath 26 to the pusher handle (not shown), and primary elongated member 16 extends proximally within secondary elongated member 18 to the pusher handle (not shown). It is at the pusher handle location that primary elongated member 16 and secondary elongated member 18 are axially manipulated relative to one another.

Secondary elongated member 18 includes a pilot portion 28 proximally adjacent proximal anchor 20P to facilitate movement of anchor 20P into outer sheath 26. Pilot portion 28 is tapered toward its relatively smaller proximal end from a relatively larger cross section having an effective diameter greater than the effective diameter of anchor 20P. The shape of pilot portion 28 is not limited to hexagonal, as represented in FIGS. 1A-1C, and may consist of a variety of shapes that taper to facilitate movement of anchor 20P into outer sheath 26 (i.e., to prevent prongs 22 from getting caught on outer sheath 26 as anchor 20P is moved into outer sheath 26).

In use, delivery system 10 is initially in its pre-insertion configuration (not shown). More specifically, primary elongated member 16, secondary elongated member 18, proximal anchor 20P, distal anchor 20D, and prosthesis 12 are all loaded within outer sheath 26 such that only pilot tip 14 is protruding from outer sheath 26. In this configuration, delivery system 10 is inserted into the body lumen (not shown).

Outer sheath 26 is proximally retracted to expose prosthesis 12 while prosthesis 12 is maintained in its radially compressed configuration by anchors 20P, 20D, as illustrated in FIG. 1A.

Secondary elongated member 18 is distally advanced to disengage the proximal end 12P of prosthesis 12 from proximal anchor 20P to allow expansion of proximal end 12P of prosthesis 12, as illustrated in FIG. 1B. More specifically, distal movement of secondary elongated member 18 causes prongs 22 to disengage apertures 24 of prosthesis 12, thereby releasing the compressive reaction force applied to the proximal end 12P of prosthesis 12 and allowing it to self-expand.

Primary elongated member 16 is proximally retracted to disengage the distal end 12D of prosthesis 12 from distal anchor 20D to allow expansion of the distal end 12D of prosthesis 12, as illustrated in FIG. 1C. More specifically, proximal movement of primary elongated member 16 causes prongs 22 to disengage apertures 24 of prosthesis 12, thereby releasing the compressive force applied to the distal end 12D of prosthesis 12 and allowing it to self-expand.

Proximal anchor 20P and distal anchor 20D are secured inside outer sheath 26 (not shown). More specifically, secondary elongated member 18 is typically proximally retracted into outer sheath 26. As explained above, the tapered shape of pilot portion 28 facilitates movement of anchor 20P into outer sheath 26 by preventing prongs 22 of proximal anchor 20P from getting caught on outer sheath 26 as anchor 20P is moved into outer sheath 26. Primary elongated member 16 is also typically proximally retracted into outer sheath 26. Because prongs 22 of distal anchor 20D extend toward the distal end 10D of delivery system 10 (i.e., away from outer sheath 26), distal anchor 20D slides easily into outer sheath 26.

Delivery system 10 is returned to its pre-insertion configuration (described above) but without prosthesis 12, and is removed from the body lumen (not shown).

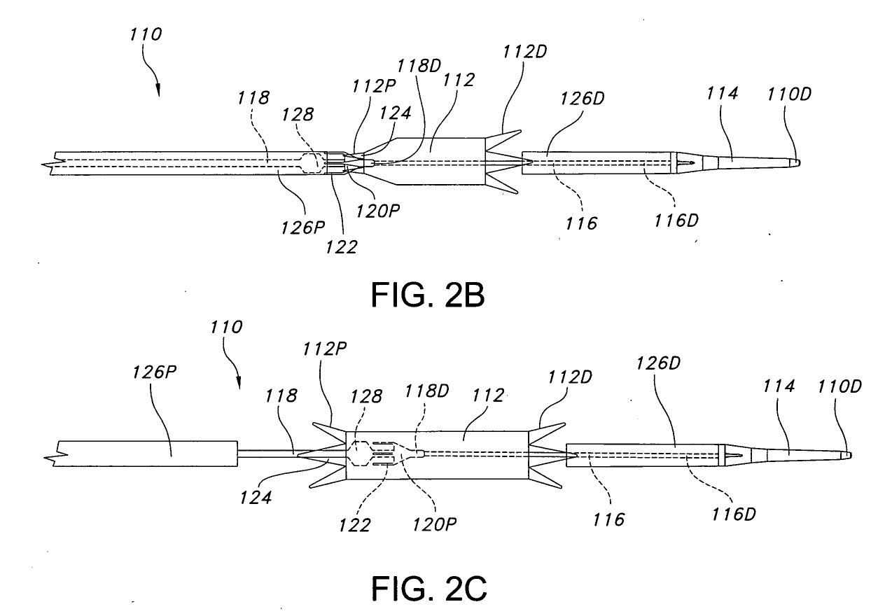

FIGS. 2A-2B illustrate an alternative exemplary configuration of a delivery system 110 for deploying a prosthesis 112 in a body lumen (not shown). A notable difference from the system shown in FIGS. 1A-1C, however, is that delivery system 110 includes only one anchor, proximal anchor 120P.

As in the system of FIGS. 1A-1C, delivery system 110 includes prosthesis 112 having a proximal end 112P, a distal end 112D, and a radially compressed configuration, as illustrated in FIG. 2A. Delivery system 110 has a proximal end (not shown) and a distal end 110D. A tip 114 is attached or over molded at the distal end 110D of delivery system 110, and a pusher handle (not shown) is located at the proximal end and remains outside the body lumen.

Delivery system 110 includes a primary elongated member 116 positioned coaxially within prosthesis 112 and having a proximal end (not shown) and a distal end 116D. A secondary elongated member 118 surrounds a portion of primary elongated member 116 and a portion of secondary elongated member 118 is positioned coaxially within prosthesis 112. Secondary elongated member 118 has a proximal end (not shown) and a distal end 118D.

Delivery system 110 further includes a proximal anchor 120P attached to secondary elongated member 118. Proximal anchor 120P is adapted for engagement with the proximal end of prosthesis 112P, thereby maintaining the proximal end 112P of prosthesis 112 in its radially compressed configuration. As described above with reference to delivery system 10 of FIGS. 1A-1C, proximal anchor 120P includes prongs 122 adapted for engagement with apertures 124 in prosthesis 112. Prongs 122 of proximal anchor 120P extend toward the proximal end of delivery system 110.

As described above with reference to delivery system 10 of FIGS. 1A-1C, prosthesis 112 may consist of, among other things, a self-expanding stent or a self-expanding stent-graft (as represented in FIGS. 2A-2C). For clarity purposes, the stent portion covered by the graft of stent-graft 112 is not shown. As illustrated in FIGS. 2A and 2B, prongs 122 of proximal anchor 120P are hooked through (i.e., engaged with) openings 124 within wire end loops. 112P and 112D, respectively, of the stent of prosthesis 112. Such engagement of anchor 120P with end 112P of prosthesis 112 maintains the proximal end 112P of prosthesis 112 in its radially compressed configuration. In other words, prongs 122 of anchor 120P effectively grab the proximal end 112P of prosthesis 112 to prevent the proximal end 112P of prosthesis 112 from self-expanding.

A distal outer sheath 126D mates with a proximal outer sheath 126P, as illustrated in FIG. 2A. Distal outer sheath 126D is adapted to be advanced to expose prosthesis 112 and allow expansion of distal end 112D of prosthesis 112, while proximal end 112P of prosthesis 112 is maintained in its radially compressed configuration, as illustrated in FIG. 2B. The radial expansion forces exerted by the distal portion of prosthesis 112 on distal outer sheath 126D create a distal frictional force exerted on prosthesis 112 as distal outer sheath 126D is advanced, thereby putting prosthesis 112 in tension against anchor 120P, which tends to reduce the radial expansion force and attendant friction. Accordingly, this design minimizes the force required to advance the sheath relative to a system without anchor 120P.

Primary elongated member 116 and secondary elongated member 118 are axially movable relative to one another to disengage prosthesis 112 from anchor 120P and permit expansion of the radially compressed proximal end 112P of prosthesis 112, as illustrated in FIG. 2C. As described above with reference to delivery system 10 of FIGS. 1A-1C, each of primary elongated member 116 and secondary elongated member 118 comprises a hypotube or single lumen extrusion. Primary elongated member 116 may guide delivery system 110 through the body lumen (not shown) over a guidewire (not shown) to the area to be repaired.

For clarity purposes, primary elongated member 116 is not shown within proximal outer sheath 126P. Similarly, secondary elongated member 118 is not shown within proximal outer sheath 126P in FIG. 2C. It is to be understood, however, that secondary elongated member 118 extends proximally within proximal outer sheath 126P to the pusher handle (not shown), and primary elongated member 116 extends proximally within secondary elongated member 118 to the pusher handle (not shown). It is at the pusher handle location that primary elongated member 116 and secondary elongated member 118 are axially manipulated relative to one another.

As described above with reference to delivery system 10 of FIGS. 1A-1C, secondary elongated member 118 includes a pilot portion 128 proximally adjacent proximal anchor 120P to facilitate movement of anchor 120P into proximal outer sheath 126P.

In use, delivery system 110 is initially in its pre-insertion configuration, as shown in FIG. 2A. More specifically, a portion of primary elongated member 116, proximal anchor 120P, and prosthesis 112 are loaded within distal outer sheath 126D with pilot tip 114 protruding from distal outer sheath 126D. A substantial portion of secondary elongated member 118 is loaded within proximal outer sheath 126P. Proximal outer sheath 126P and distal outer sheath 126D are mated. In this configuration, delivery system 110 is inserted into the body lumen (not shown).

Distal outer sheath 126D is distally advanced away from mating proximal outer sheath 126P to expose prosthesis 112 to allow expansion of the distal end 112D of prosthesis 112 while the proximal end 112P of prosthesis 112 is maintained in its radially compressed configuration by proximal anchor 120P, as illustrated in FIG. 2B.

Secondary elongated member 118 is distally advanced to disengage the proximal end 112P of prosthesis 112 from proximal anchor 120P to allow expansion of the proximal end 112P of prosthesis 112, as illustrated in FIG. 2C.

Proximal anchor 120P is secured inside proximal outer sheath 126P (not shown). More specifically, secondary elongated member 118 is typically proximally retracted into proximal outer sheath 126P. As explained above with reference to delivery system 10 of FIGS. 1A-1C, the tapered shape of pilot portion 128 facilitates movement of anchor 120P into proximal outer sheath 126P by preventing prongs 122 from getting caught on proximal outer sheath 126P as anchor 120P is moved into proximal outer sheath 126P. Primary elongated member 116 is also typically proximally retracted into proximal outer sheath 126P.

Distal outer sheath 126D is mated with proximal outer sheath 126P. Delivery system 110 is, returned to its pre-insertion configuration (described above) but without prosthesis 112, and is removed from the body lumen (not shown).

An exemplary material for forming primary elongated member 16, 116, secondary elongated member 18, 118, proximal anchor 20P, 120P, distal anchor 20D, and prongs 22 is stainless steel. The present invention, however, is not limited to this material, and may include any materials, including, for example, metallic (titanium, for example) or non-metallic (a polymer or other composite material, for example) materials that offer desired properties including both strength and flexibility.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A delivery system for deploying a prosthesis in a body lumen, the prosthesis having a proximal end, a distal end, and a radially compressed configuration, said delivery system having a proximal end and a distal end and comprising:
a primary elongated member positioned coaxially within the prosthesis, said primary elongated member comprising a proximal end and a distal end;
a secondary elongated member surrounding a portion of said primary elongated member, a portion of said secondary elongated member positioned coaxially within the prosthesis, said secondary elongated member comprising a proximal end and a distal end;
a proximal anchor attached to said secondary elongated member, said anchor adapted for engagement with the proximal end of the prosthesis, thereby maintaining the prosthesis in its radially compressed configuration; and
at least one outer sheath adapted to be retracted to expose the prosthesis while the prosthesis is maintained in its radially compressed configuration,
wherein said primary elongated member and said secondary elongated member are axially movable relative to one another to disengage the prosthesis from said anchor and permit expansion of the radially compressed prosthesis.

2. The delivery system of claim 1 wherein said proximal anchor comprises prongs adapted for engagement with apertures in the prosthesis.

3. The delivery system of claim 2 wherein said prongs of said proximal anchor extend toward said proximal end of said delivery system.

4. The delivery system of claim 1 further comprising a distal anchor attached to said primary elongated member.

5. The delivery system of claim 4 wherein each of said proximal anchor and said distal anchor comprises prongs adapted for engagement with apertures in the prosthesis.

6. The delivery system of claim 5 wherein said prongs of said proximal anchor extend toward said proximal end of said delivery system, and said prongs of said distal anchor extend toward said distal end of said delivery system.

7. The delivery system of claim 1 wherein said secondary elongated member further comprises a pilot portion proximally adjacent said proximal anchor to facilitate movement of said proximal anchor into said outer sheath.

8. The delivery system of claim 7 wherein said pilot portion is tapered toward a relatively smaller proximal end from a relatively larger cross section.

9. The delivery system of claim 1 wherein each of said primary elongated member and said secondary elongated member comprises a hypotube or single lumen extrusion.

10. The delivery system of claim 1 wherein the prosthesis comprises a stent or a stent-graft.

11. The delivery system of claim 1 wherein the prosthesis comprises a stent having an uncovered portion at its proximal end for receiving said proximal anchor.

12. The delivery system of claim 4 wherein the prosthesis comprises a stent having an uncovered portion at its distal end for receiving said distal anchor.

13. The delivery system of claim 4 wherein the prosthesis comprises a stent having an uncovered portion at each of its proximal end and its distal end for receiving said proximal anchor and said distal anchor, respectively.

14. A method for deploying a prosthesis in a body lumen, the prosthesis having a proximal end, a distal end, and a radially compressed configuration, said method comprising the steps of:
(a) inserting a delivery system into the body lumen, the delivery system having proximal end and a distal end and comprising
a primary elongated member positioned coaxially within the prosthesis, the primary elongated member having a proximal end and a distal end,
a secondary elongated member surrounding a portion of the primary elongated member, a portion of said secondary elongated member positioned coaxially within the prosthesis, the secondary elongated member having a proximal end and a distal end,
a proximal anchor attached to the secondary elongated member and engaged with the proximal end of the prosthesis and a distal anchor attached to the primary elongated member and engaged with the distal end of the prosthesis, and
an outer sheath;
(b) proximally retracting the outer sheath to expose the prosthesis while the prosthesis is maintained in its radially compressed configuration under tension between the proximal and distal anchors; and
(c) distally advancing the secondary elongated member to disengage the proximal end of the prosthesis from the proximal anchor to allow expansion of the proximal end of the prosthesis.

15. The method of claim 14 further comprising the steps of:
(d) proximally retracting the primary elongated member to disengage the distal end of the prosthesis from the distal anchor to allow expansion of the distal end of the prosthesis;
(e) securing the proximal anchor and the distal anchor inside the outer sheath; and
(f) removing the delivery system from the body lumen.

16. A method for deploying a prosthesis in a body lumen, the prosthesis having a proximal end, a distal end, and a radially compressed configuration, said method comprising the steps of:
(a) inserting a delivery system into the body lumen, the delivery system having a proximal end and a distal end and comprising
a primary elongated member positioned coaxially within the prosthesis, the primary elongated member having a proximal end and a distal end,
a secondary elongated member surrounding a portion of the primary elongated member, a portion of the secondary elongated member positioned coaxially within the prosthesis, the secondary elongated member having a proximal end and a distal end,
a proximal anchor attached to the secondary elongated member and engaged with the proximal end of the prosthesis,
a proximal outer sheath, and
a distal outer sheath mated with the proximal outer sheath;
(b) distally advancing the distal outer sheath away from the mating proximal outer sheath to expose the prosthesis to allow expansion of the distal end of the prosthesis while the proximal end of the prosthesis is maintained in its radially compressed configuration; and
(c) distally advancing the secondary elongated member to disengage the proximal end of the prosthesis from the proximal anchor to allow expansion of the proximal end of the prosthesis.

17. The method of claim 16 further comprising the steps of:
(d) securing the proximal anchor inside the proximal outer sheath;
(e) mating the distal outer sheath with the proximal outer sheath; and
(f) removing the delivery system from the body lumen.
